(19) United States
(12) Patent Application Publication
(10) Pub. No.: US 2023/0069469 A1
(43) Pub. Date: Mar. 2, 2023

(54) PLASTICIZER COMPOSITION AND PROCESS TO PRODUCE A PLASTICIZER COMPOSITION

(71) Applicant: CARGILL, INCORPORATED, Wayzata, MN (US)

(72) Inventors: Oreste Valdir BARALDI FILHO, Jandira - Sao Paulo (BR); Fabio Angelo BRUCOLI, Sao Paulo - Sao Paulo (BR); Marcos Cesar FERNANDEZ, Santo Andre - Sao Paulo (BR); Todd L. KURTH, Maple Grove, MN (US); Alzirio NICOLETTI, Rio Claro - Sao Paulo (BR); Luciana PEREIRA AMORIM, Reneville - Mairinque (BR); Antonio ROQUE DUARTE, Jr., São Roque - São Paulo (BR); Christopher Patrick STEVERMER, St. Louis Park, MN (US)

(73) Assignee: CARGILL, INCORPORATED, Wayzata, MN (US)

(21) Appl. No.: 17/759,657

(22) PCT Filed: Jan. 28, 2021
(86) PCT No.: PCT/US2021/015395

§ 371 Date: Jul. 28, 2022

Related U.S. Application Data
(60) Provisional application No. 62/967,772, filed on Jan. 30, 2020.
(60) Provisional application No. 63/122,607, filed on Dec. 8, 2020.

(51) Int. Cl. C08K 5/1515; C08K 5/11; C08K 5/00; C07D 303/42; C08F 114/06

(52) U.S. Cl. CPC C08K 5/1515; C08K 5/11; C08K 5/0016; C07D 303/42; C08F 114/06

(57) ABSTRACT

Plasticizer compositions are prepared by reacting a vegetal oil with isopentyl alcohol to provide a glyceride/fatty ester composition comprising from about from about 40 to about 80% wt isopentyl fatty acid ester, and reacting the glyceride/fatty ester composition with acetic anhydride to form an acetylated glyceride/fatty ester composition. The vegetal oil may be epoxidized before the reaction with isopentyl alcohol, or the acetylated glyceride/fatty ester composition first prepared and then epoxidized. Epoxidized acetylated glyceride/fatty ester plasticizer compositions are also described. Alternatively, plasticizer compositions are prepared by preparing a C4-C10 fatty ester composition separately from acetylated glyceride composition and mixing the compositions in a weight ratio of 3:7 to 7:3. The C4-C10 fatty ester composition and the acetylated glyceride composition may be epoxidized either before or after mixing.

### CROSS-REFERENCE TO RELATED APPLICATIONS

This application claims the benefit of U.S. Provisional Patent Application No. 62/967,772, filed Jan. 30, 2020 and U.S. Provisional Patent Application No. 63/122,607, filed Dec. 8, 2020, each of which is hereby incorporated by reference herein in its entirety.

### FIELD

The present invention relates to a new plasticizer composition and to a process to produce a plasticizer composition, which is a partial transesterification process. The present invention also relates to the use of the plasticizer composition, to PVC compounding comprising the mentioned plasticizer composition and to PVC articles comprising the PVC compounder.

### BACKGROUND

By definition, plasticizers are compounds or chemical substances added to a polymer matrix in order to impart flexibility, processability-workability, lubricity, softness or elasticity.

Plasticizers are used in elastomers and plastics articles, such as in PVC (Polyvinyl Chloride), EVA (Ethylene vinyl acetate) and PU (Polyurethanes), among others, in many application materials as adhesives and sealants, films and sheets, flooring and wall covering, laminates, hoses, footwear, coated fabric materials (i.e. synthetic leathers and tarpaulins), childcare articles (i.e. toys), wires and cables, medical devices (e.g.: tubing, bags) among others.

The choice of a plasticizer is an important factor in the formulation. When selecting a product, the formulator considers the significant qualities of the plasticizer, such as compatibility, permanence, efficiency and, of course, cost.

The use of plasticizers allows the manufacturer of vinyl compounds to balance the multiple required characteristics of its compound. Originally, plasticizers were used to transform rigid polyvinyl chloride (PVC) resins into flexible products, reducing their toughness.

The Polyvinyl chloride (PVC) products are divided into two main classes: rigid and flexible. Plasticizers have been used for many years in the production of flexible PVC for a wide variety of applications. The level and type of plasticizer used is selected to obtain the characteristics required for each application. They are generally colorless and odorless, relatively non-volatile liquids and exhibiting low solubility in water. They are mostly esters or polyesters, including others based on adipic, phosphoric, sebaceous, trimetylic or azelatic acids.

The plasticizers that have high compatibility with PVC are regarded as primary plasticizers and those with limited compatibility, as secondary plasticizers, this last ones used as co-additives to complement a specific material performance (for instance, ESO is largely used as secondary plasticizer to impart improved thermal stability for the PVC articles).

The plasticizers are divided according to their chemical characteristics: Phthalate: DIBP (di iso butyl phthalate), DOP (dioctyl phthalate), DIDP (di iso decyl phthalate), DINP (diisononyl phthalate), DEHP (di(2-etiyhexyl) phthalate); Adipates: DOA (dioctyl adipate); Azelatos: DOZ (dioctyl azelate); Triesters: TOTM (trioctyl trimellitate); Polyesters: polymeric plasticizers; Epoxidized: OSE (epoxidized soybean oil); Phosphate: TCP (tricresyl phosphate); and Dioctyl cyclohexanoate (DOCH). The phthalic plasticizers (DOP, DIBP, DIDP and DINP) are the traditional and most widely used because of the favorable cost and performance balance.

The secondary plasticizer most used is ESO (epoxidized soybean oil), which also acts as auxiliary in the thermal stabilization of PVC together with stabilizers based on salts of barium, cadmium and zinc.

Polyvinyl chloride (PVC) is one of the most consumed plastic in the world and combined with the consumption of this polymer is that of its additives, among which can be highlighted the plasticizers. For PVC, the phthalate class is still the most used, and of these, dioctyl phthalate (DOP) and the diisononyl phthalate (DINP) have been considered the standard and used for multi-purpose plasticizers for PVC.

DOP (di octyl phthalate) is used in blood bags, human tissue simulators, packaging, shoes, tubes and profiles, etc. The DOP represents more than 50% of the total plasticizers produced in the world.

An additional common use of plasticizers is in production of plastisol. Plastisol is an emulsion of PVC or other polymer particles in a liquid plasticizer. Aside from molding, plastisol is also used in specific manufacturing process commonly used for the production of synthetic leather, tarpaulins, coated fabric articles, flooring and toys, among others.

Despite all the positive aspects, the whole class of phthalates, in the last decades, have undergone through extensive testing for evaluating regarding possible health and environmental effects. To date, four classified low orthophthalates—DOP, DBP, DIBP and BBP—have been found to have adverse endocrine-related effects in laboratory animal studies with specific thresholds.

In some cases, data on the extraction of phthalate from the polymeric matrix were confirmed by extraction, making it impossible to apply it in some situations, especially when in direct contact with food. Overall the regulatory agencies, especially in Europe and North America, have limited the use of these substances in articles which will potentially be in contact with human for a prolonged time skin and for childcare articles.

There are four (4) main forms of loss of plasticizer from a plasticized polymer:
Volatilization: loss of plasticizer from the material to the atmosphere;
Extraction: loss of plasticizer from the material for liquids (oils, water, greases among other agents). In this case, the higher the molecular weight of the plasticizer and the lower its chemical affinity with the liquid the greater its resistance to extraction;
Migration: loss of plasticizer by transfer between two surfaces that are in contact; and
Exudation: loss of plasticizer by emission from the material.

Accordingly, the plasticizer's market today faces many regulatory issues, mainly the limited use of the phthalates. There are many restrictions for the use of phthalates. The restrictions limits for childcare articles, materials which may have prolonged contact with the human skin and others which may be in contact with food, for instance, are stricter.

Additionally, recently, the adipose tissue has been recognized as a true endocrine organ, and a subset of EDCs have been named Metabolism Disrupting Chemicals (MDCs) because of their ability to promote adiposity and alteration of energy homeostasis. MDCs include plasticizers such as bisphenol (BPA) and certain phthalates used in PVC plastics, in outdoor applications (roofs, furniture) and dip-coating.

With that, it has been an increasing demand and interest by the market and consumers looking for alternative plasticizers which can replace the phthalates and be in compliance with the global regulatory legislation. The so-called biobased plasticizers, made from modified natural based feedstocks such as vegetable oils, fatty acids and their derivatives, have been identified as feasible options and viable solutions to help the phasing out of these regulated petro-based substances.

There are many processes known from the prior art to prepare compositions that are useful as plasticizers. Such methods include esterification, interesterification, trans-alcoholysis or trans-esterification.

Transesterification (see illustration below) is a process of exchanging the organic group R″ of an ester with the organic group R′ of an alcohol, resulting in different alcohol and ester. These reactions are often catalyzed by the addition of an acid or base catalyst.

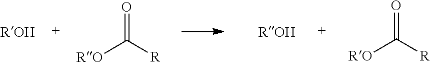

A total transesterification of a vegetable oil means that 1 mol of the oil reacts with 3 moles of alcohol. In the partial transesterification, less than 3 mol of alcohol is used in relation to the oil.

U.S. Pat. No. 8,865,936 (corresponding to Brazilian patent application PI 0704776) filed by SGS Polímeros Ltda, U.S. Pat. No. 8,865,936 describes reacting epoxidized glycerol fatty esters and ethyl acetate to form epoxidized acetylated monoglycerides and epoxidized fatty acid ethyl ester.

U.S. Pat. No. 8,623,947 (corresponding to Brazilian patent application PI 0705276), from Nexoleum Bioderivados Ltda, a Brazilian company, concerns the partial transesterification of a vegetable oil performed with ethanol or glycerin, followed by acetylation and epoxidation.

U.S. Pat. No. 9,303,140 (corresponding to Brazilian patent application PI 0705621), describes PVC plasticizers composed of epoxidized bioesters of vegetable oil fatty acids obtained by partial transesterification with an alcohol, and glycerin and further acetylation and epoxidation. See the abstract. The objective as described at column 2, lines 23-27 is to prepare technically and economically viable alternatives of primary plasticizers for PVC compounds derived exclusively from renewable sources (vegetable oils and sugar cane ethanol) that are completely compatible with the PVC resin.

EP 2070980A2 describes primary PVC plasticizers composed of epoxidized ethyl and/or isoamyl (i.e. isopentyl) esters of vegetable oil fatty acids and to the compounds of PVC plasticized with epoxidized bioesters, belonging to the technical field of polymer additives, developed from renewable sources such as vegetable oils and sugar cane, to reduce the cost and improve the properties of PVC compounds. See the abstract. Isoamylic alcohol specifically described as being obtained from the residue of sugar cane based ethanol production (also known as fusel oil). See paragraph [0018]. In the preferred embodiments, vegetable oils completely transesterified with the alcohols, e.g., isoamylic alcohol, and later epoxidized. See paragraph [0032].

WO 2012/174620 (corresponding to Brazilian patent application PI 1102794, filed by KEKAPAR Adm. Part. S.A.) claims “a composition with primary plasticizers without phthalate characterized by comprising vegetable oil derivatives, said derivatives resulting from the esterification of epoxidized vegetable oils, mainly monoacetates of epoxidized vegetable oil.” The process to obtain such a composition is to react an epoxidized vegetable oil, comprising mainly a monoacetate of epoxidized vegetable oil, with triacetin.

### SUMMARY

In view of the issues involving the phthalate compounds and currently known alternative plasticizers, there is a need for an effective plasticizer developed from renewable sources (i.e. “green”) with good performance that is not based on phthalate plasticizers.

In an aspect, a plasticizer composition is prepared by a sequential process, wherein a number of reactions are carried out in series to further modify successive reaction products from a single vegetal oil/fatty acid source starting material. This type of sequential process may be stylized as a “one-pot” process, although the sequential reactions may optionally be carried out in different reaction vessels depending on availability and convenience at the processing facility.

In an aspect, a process to produce a plasticizer composition comprises
a) reacting a vegetal oil with isopentyl alcohol to provide a glyceride/fatty ester composition comprising from about from about 40 to about 80% wt isopentyl fatty acid ester;
b) reacting the glyceride/fatty ester composition of step a) with acetic anhydride to form an acetylated glyceride/fatty ester composition; and
c) epoxidizing the acetylated glyceride/fatty ester composition of step b) to form an epoxidized acetylated glyceride/fatty ester plasticizer composition;
wherein the epoxidized acetylated glyceride/fatty ester plasticizer composition is free of phthalate.

In an aspect, a process to produce a plasticizer composition comprises
a) epoxidizing a vegetal oil to form an epoxidized vegetal oil;
b) reacting the epoxidized vegetal oil of step a) with isopentyl alcohol to provide an epoxidized glyceride/fatty ester composition comprising from about from about 40 to about 80% wt isopentyl fatty acid ester; and
c) reacting the epoxidized glyceride/fatty ester composition of step b) with acetic anhydride to form an epoxidized acetylated glyceride/fatty ester composition;
wherein the epoxidized acetylated glyceride/fatty ester plasticizer composition is free of phthalate.

In an aspect, a plasticizer composition is prepared by a parallel process, wherein two or more single vegetal oil/fatty acid source starting materials are reacted in parallel, with subsequent mixing of product components to provide the desired plasticizer composition. This type of parallel process may be stylized as a “two-pot” process, although the number of reaction vessels actually used may vary depending on availability and convenience at the processing facility.

In an aspect, a process to produce an epoxidized acetylated glyceride/fatty ester plasticizer composition, comprising:

a) reacting a C4-C10 alcohol with an alkyl ester of an unsaturated fatty acid to provide a C4-C10 fatty ester composition;

b) reacting a vegetal oil with glycerin to provide a mixture of monoacyl glycerides, diacyl glycerides, triacyl glycerides, residual glycerin and fatty acid;

c) reacting the mixture of monoacyl glycerides (MAG), diacyl glycerides (DAG), and triacyl glycerides (TAG) composition of step b) with acetic anhydride to form an acetylated glyceride composition comprising from about 7 to about 35% wt di-acylated monoglycerides and from about 45 to about 60% w mono-acylated diglycerides; and

d) mixing the C4-C10 fatty ester composition with the acetylated glyceride composition in a weight ratio of 3:7 to 7:3;

wherein the C4-C10 fatty ester composition and the acetylated glyceride composition are epoxidized either before mixing step d) or after mixing step d) to provide an epoxidized acetylated glyceride/fatty ester plasticizer composition

In an aspect, an epoxidized acetylated glyceride/fatty ester plasticizer composition is provided that is prepared by any of the processes as described herein.

In an aspect, an epoxidized acetylated glyceride/fatty ester plasticizer composition comprises
i) from about 40-80% wt of epoxidized fatty acid isopentyl ester and/or its isomers
ii) from about 5 to about 25% wt of acetylated and epoxidized monoglyceride or its isomers;
ii) from about 10 to about 30% wt of acetylated and epoxidized diglyceride and/or its isomers;
iii) from about 3 to about 20% wt of epoxidized triglyceride;
iv) from about 0 to about 10% wt of epoxidized fatty acid ethyl ester;
v) from about 0 to about 10% wt of epoxidized fatty acid methyl ester; and
vi) from about 0 to about 8% wt of triacetin.

In an aspect, a plasticized polyvinyl chloride composition comprises any of the epoxidized acetylated glyceride/fatty ester plasticizer composition as described herein.

In an aspect, of either of the sequential processes as described above, the steps a-c are carried out in a sequential manner without intermediate steps, which minimizes processing steps and provides for a simplified process without expensive and/or time consuming intermediate work-up steps between the stated steps. In an aspect, the compositions are not compositionally altered between the steps except to optionally add non-reactive components such as solvents. In an aspect, the subsequent steps are carried out in the same reaction vessel, so that the processes may be referred to as “one-pot” processes. It has been discovered that by selection of the starting materials and carrying out the reactions as described, a plasticizer product can be prepared with simple ingredients that are highly compatible with, for example, PVC, and which are effective a plasticizers.

In an aspect, the process wherein the vegetal oil is reacted with isopentyl alcohol and the glyceride/fatty ester composition is reacted with acetic anhydride to form an acetylated glyceride/fatty ester composition before epoxidation provides particular benefit, because potentially undesirable side reactions such as formation of estolides, formation of undesired hydroxyl functionality and adverse color generation may be avoided.

It has been found that plasticizers prepared as described herein can exhibit superior plasticizing efficiency. In an aspect, plasticizers as described herein achieve the same plasticizing effect (e.g. keep the same PVC sheet hardness) while reducing the plasticizer content of the PVC material up to 10% as compared to DINP and DOCH plasticizers. Likewise, the mechanical properties of the final PVC product, such as Tensile Strength, Elongation and Elastic Modulus were kept similar to slightly better performance while reducing the plasticizer content of the PVC material up to 10% as compared to DINP and DOCH plasticizers.

Additionally, it has been found that plasticizers as described herein can exhibit differentiated and significant improvement in performance parameters measured by the hardness, density and mass loss in dry-blend formats for use in suspension PVC applications and/or in emulsions for use in plastisol PVC applications.

It further has been found that plasticizers as described herein can exhibit excellent low exudation properties, even in final applications comprising greater than 50 PHR, or greater than 70 PHR, or greater than 80 PHR levels. Likewise, plasticizers as described herein can exhibit excellent volumetric resistivity characteristics in wire coating applications.

These beneficial properties are achieved in a plasticizer composition comprising high content of renewable sourced ingredients. The source ingredients for the present plasticizer composition are readily available, and the resulting plasticizer composition may be prepared at a cost competitive price.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this application, illustrate several aspects of the invention and together with a description of the embodiments serve to explain the principles of the invention. A brief description of the drawings is as follows:

FIG. 1 illustrates one of the reaction steps of an aspect of the present process.

FIG. 2 illustrates one of the reaction steps of an aspect of the present process.

FIG. 3 illustrates one of the reaction steps of an aspect of the present process.

FIG. 4 illustrates one of the reaction steps of an aspect of the present process.

FIG. 5 illustrates one of the reaction steps of an aspect of the present process.

### DETAILED DESCRIPTION

The aspects of the present invention described below are not intended to be exhaustive or to limit the invention to the precise forms disclosed in the following detailed description. Rather a purpose of the aspects chosen and described is by way of illustration or example, so that the appreciation and understanding by others skilled in the art of the general principles and practices of the present invention can be facilitated.

The vegetal oil used in the present process in an aspect is selected from the group consisting of soybean oil, canola oil, rapeseed oil, sunflower oil, linseed oil, corn oil, and mixtures thereof. In an aspect, the vegetal oil is selected from the group consisting of soybean oil, canola oil, rapeseed oil, and mixtures thereof. In an aspect, the vegetal oil is soybean oil. in an aspect, the vegetal oil is canola oil. In an aspect, the vegetal oil is rapeseed oil. It should be noted that canola oil is a vegetable oil derived from a variety of rapeseed that is low in erucic acid. In contrast, rapeseed oil comprises appreciable amounts of erucic acid.

In an aspect, the vegetal oil used in the present process has an Iodine Value of from about 80 to about 140 cg I/g. For purposes of the present discussion, Iodine Value is determined by the AOCS-Cd 1b-87 test method.

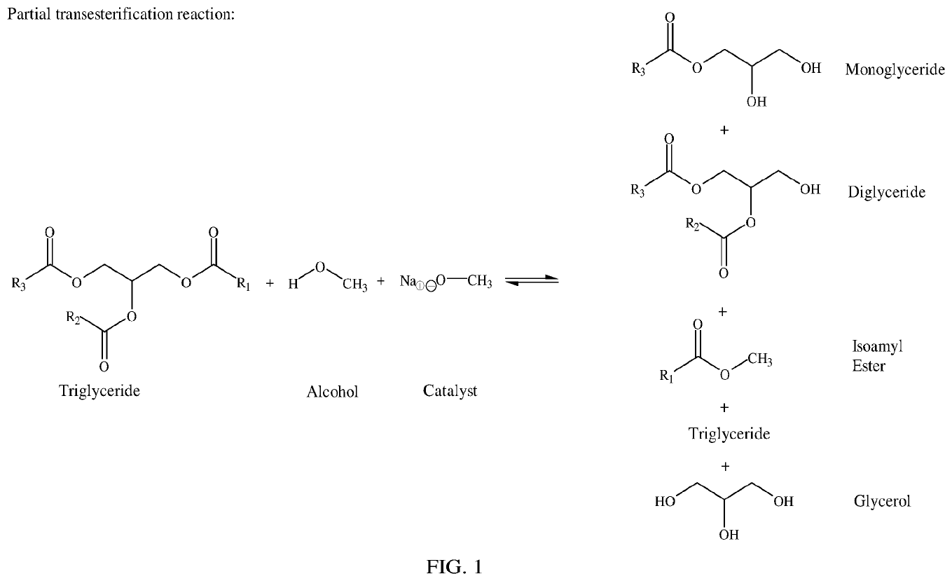

Turning now to the sequential or “one-pot” processes, in an aspect, the vegetal oil is reacted with isopentyl alcohol prior to epoxidation to provide a glyceride/fatty ester composition comprising from about from about 40 to about 80% wt isopentyl fatty acid ester. This reaction is carried out as a partial transesterification, as shown in FIG. 1.

Isopentyl alcohol as used in this reaction is typically a byproduct of ethanol refining (for example, from sugar cane or corn), and is thus a bio renewable and less expensive source.

The partial transesterification reaction can be catalyzed by basic and acids catalysts, such as sodium or potassium hydroxides and alkoxides, and sulfuric and hydrochloric acids. Examples of catalysts include sodium methylate (Na(CH₃O)), methanesulfonic acid and phosphoric acid. In an aspect, the partial transesterification is carried out under a nitrogen sparge.

In an aspect, the partial transesterification is carried out as a single reaction stage that optionally comprises alcohol stripping conditions to remove any methanol or ethanol present in the reaction composition. In an aspect, the partial transesterification is carried out as a single reaction stage at a reaction temperature of from about 30° C. to about 80° C. In an aspect, the transesterification reaction takes place at about 65° C. and the reaction time is about 20 min to about 5 hours.

In an aspect, the partial transesterification is carried out as a first reaction stage, followed by a second reaction stage that optionally comprises alcohol stripping conditions to remove any methanol or ethanol present in the reaction composition.

In an aspect, the partial transesterification is carried out as a first reaction stage that takes place at a temperature of from about 30° C. to about 80° C. In an aspect, the partial transesterification first reaction stage takes place at a temperature of about 65° C. In an aspect, the partial transesterification first reaction stage reaction time is from about 20 min to about 5 hours. In an aspect, the partial transesterification first reaction stage reaction time is from about 2 to about 2.5 hours.

In an aspect, the partial transesterification second reaction stage takes place at a temperature of from about 30° C. to about 80° C. In an aspect, the partial transesterification second reaction stage takes place at a temperature of about 65° C. In an aspect, the partial transesterification second reaction stage reaction time is from about 20 min to 5 hours. In an aspect, the partial transesterification second reaction stage reaction time is from about 1 to about 1.5 hours.

In an aspect, progress of the partial transesterification reaction is monitored measured by gas chromatography, and the reaction is terminated when the desired ester content of the final product is achieved.

In aspect, after the partial transesterification is completed an alcohol stripping step is carried out to remove any methanol, ethanol or isopentyl alcohol present in the reaction composition. In an aspect, the alcohol stripping step is carried out at a temperature of from about 40° C. to about 150° C. In an aspect, the alcohol stripping step is carried out at a temperature of from about 80° C. to about 130° C. In an aspect, the alcohol stripping step reaction time is from about 20 minutes to 5 hours. In an aspect, the alcohol stripping step reaction time is from about 30 minutes to about 4.5 hours. In an aspect, the alcohol stripping step is performed in vacuum. In an aspect, the alcohol stripping step is performed in vacuum, wherein nitrogen is injected through the bottom of the reactor and temperature is applied. In an aspect, the alcohol stripping step under vacuum is carried out at a temperature of from about 85° C. to about 95° C. for a time of from about 1 to 3 hours.

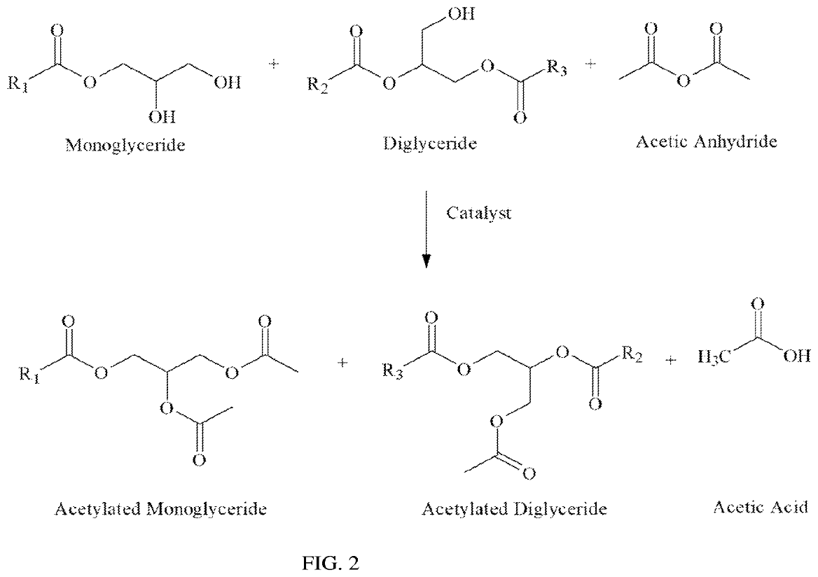

The resulting glyceride/fatty ester composition is reacted with acetic anhydride in the presence of a catalyst to form an acetylated glyceride/fatty ester composition. This acetylation reaction is shown in FIG. 2.

In an aspect, the acetylation reaction is optionally catalyzed with a catalyst selected from methanosulfonic acid and sulfuric acid. In an aspect, the acetylation reaction is not catalyzed. It has been found that in some cases compositions prepared using a separate acetylation catalyst can exhibit undesirable color properties.

In an aspect, the acetylation reaction takes place at a temperature of from about 60° C. to about 120° C. In an aspect, the acetylation reaction takes place at a temperature of about 85° C. In an aspect, the acetylation reaction time is from about 1 to 8 hours. In an aspect, the acetylation reaction time is from about 2 to about 4 hours.

In aspect, the acetylation reaction is followed by an acetic acid stripping step to remove any undesired acetic acid present in the reaction composition.

In an aspect, the acetic acid stripping step is carried out at a temperature of from about 100° C. to about 180° C. In an aspect, the acetic acid stripping step is carried out at a temperature of from about 135° C. to about 150° C. In an aspect, the acetic acid stripping step reaction time is from about 1 hour to about 7 hours. In an aspect, the acetic acid stripping step reaction time is from about 2 hours to about 6 hours.

The acetylated glyceride/fatty ester composition is epoxidized by reacting with hydrogen peroxide in the presence of catalyst. In an aspect, the catalyst is selected from phosphoric acid, formic acid or acetic acid. Once all the hydrogen peroxide is added and the double bonds are consumed the product is water washed multiple times to remove residual acid and peroxide before being dried under vacuum. The resulting product is the epoxidized version of the intermediate.

In an aspect, the epoxidation reaction is carried out at a temperature of from about 50° C. to about 100° C. In an aspect, the epoxidation reaction is carried out at a temperature of from about 55° C. to about 75° C. In an aspect, the epoxidation reaction time is from about 1 to about 5 hours. In an aspect, the epoxidation reaction time is from about 1.5 to about 3 hours.

In an aspect, the vegetal oil as discussed above is first epoxidized prior to carrying out the partial transesterification reaction with isopentyl alcohol and the acetylation reaction. In this aspect, the reaction conditions are generally as described above, with the only difference being the order of the reaction steps.

In an aspect, the acetylated glyceride/fatty ester composition is additionally processed after any of the steps a), b) and c) in a further step such as bleaching, filtration, deodorization, washing and stripping.

In an aspect, a bleaching step is carried out to decrease the color of the produced product. In an aspect, a bleaching step is carried out by increasing the pH of the acetylated glyceride/fatty ester composition to a pH that is about 9 or higher.

In an aspect, no bleaching step is carried out in the process. It has been found that a bleaching step can particularly be avoided when the epoxidation step is carried out after the partial transesterification reaction and the acetylation reaction, due to the superior color performance when the steps are carried out in that order.

In an aspect, a washing step may be conducted after an acetylation step. In an aspect, a washing step may be conducted after a transesterification step. In an aspect, no washing step is carried out, because it has been found that undesirable removal of molecules, especially of monoglycerides and diglycerides, may occur.

In an aspect, the epoxidized acetylated glyceride/fatty ester plasticizer composition comprises:
i) from about 40-80% wt of epoxidized fatty acid isopentyl ester and/or its isomers
ii) from about 5 to about 25% wt of acetylated and epoxidized monoglyceride or its isomers;
ii) from about 10 to about 30% wt of acetylated and epoxidized diglyceride and/or its isomers;
iii) from about 3 to about 20% wt of epoxidized triglyceride;
iv) from 0 to about 10% wt of epoxidized fatty acid ethyl ester;
v) from 0 to about 10% wt of epoxidized fatty acid methyl ester; and
vi) from 0 to about 8% wt of triacetin.

In an aspect, the epoxidized acetylated glyceride/fatty ester plasticizer composition comprises:
i) from about 45-60% wt of epoxidized fatty acid isopentyl ester and/or its isomers
ii) from about 10 to about 20% wt of acetylated and epoxidized monoglyceride or its isomers;
ii) from about 15 to about 25% wt of acetylated and epoxidized diglyceride and/or its isomers;
iii) from about 5 to about 15% wt of epoxidized triglyceride;
iv) from 0 to about 5% wt of epoxidized fatty acid ethyl ester;
v) from 0 to about 5% wt of epoxidized fatty acid methyl ester; and
vi) from 0 to about 5% wt of triacetin.

Turning now to the parallel or “two-pot” processes, in an aspect a C4-C10 fatty ester composition is prepared by reacting a C4-C10 alcohol with an alkyl ester of an unsaturated fatty acid in a transesterification reaction a).

In an aspect, the C4-C10 alcohol is selected from isobutanol, isopentanol, 2-ethyl hexanol, isoheptanol, isooctanol, isononanol and isodecanol; and mixtures thereof. In an aspect, the C4-C10 alcohol is selected from isobutanol, isopentanol, 2-ethyl hexanol, and mixtures thereof. In an aspect, the C4-C10 alcohol is isopentanol.

In an aspect, the alkyl portion of the alkyl ester of an unsaturated fatty acid is selected from methyl, ethyl and propyl groups. In an aspect, the alkyl portion of the alkyl ester of an unsaturated fatty acid is methyl.

In an aspect, the fatty acid portion of the alkyl ester of an unsaturated fatty acid is a residue of a vegetal oil selected from the group consisting of Soybean Oil, Canola Oil, Rapeseed Oil, Sunflower Oil, Linseed oil, Corn Oil, and mixtures thereof. In an aspect, the fatty acid portion of the alkyl ester of an unsaturated fatty acid is a residue of a vegetal oil selected from the group consisting of Soybean Oil, Canola Oil, Rapeseed Oil, and mixtures thereof. In an aspect, the fatty acid portion of the alkyl ester of an unsaturated fatty acid is a residue of Soybean Oil. In an aspect, the fatty acid portion of the alkyl ester of an unsaturated fatty acid is a residue of Canola Oil. In an aspect, the fatty acid portion of the alkyl ester of an unsaturated fatty acid is a residue of Rapeseed Oil.

In an aspect, the alcohol portion of the C4-C10 fatty ester is selected from isobutyl, isopentyl, and 2-ethyl hexyl, isoheptyl, isooctyl, isononyl, isodecyl; and mixtures thereof. In an aspect, the alcohol portion of the C4-C10 fatty ester is selected from isobutyl, isopentyl, and 2-ethyl hexyl, and mixtures thereof. In an aspect, the alcohol portion of the C4-C10 fatty ester is isopentyl.

The transesterification reaction can be catalyzed by basic and acids catalysts, such as sodium or potassium hydroxides and alkoxides, and sulfuric and hydrochloric acids. Examples of catalysts include sodium methylate (Na(CH₃O)), methanesulfonic acid and phosphoric acid. In an aspect, the transesterification is carried out under a nitrogen sparge.

In an aspect, the transesterification is carried out as a single reaction stage that optionally comprises alcohol stripping conditions to remove any undesired alcohols present in the reaction composition, for example, methanol or ethanol. In an aspect, the transesterification is carried out as a single reaction stage at a reaction temperature of from about 30° C. to about 80° C. In an aspect, the transesterification reaction takes place at about 65° C. and the reaction time is about 20 min to about 5 hours.

In an aspect, the transesterification is carried out as a first reaction stage, followed by a second reaction stage that optionally comprises alcohol stripping conditions to remove any undesired alcohols present in the reaction composition.

In an aspect, progress of the transesterification reaction is monitored measured by gas chromatography, and the reaction is terminated when the desired ester content of the final product is achieved.

In an aspect, the alcohol stripping step is carried out at a temperature of from about 40° C. to about 150° C. In an aspect, the alcohol stripping step is carried out at a temperature of from about 80° C. to about 130° C. In an aspect, the alcohol stripping step reaction time is from about 20 minutes to 5 hours. In an aspect, the alcohol stripping step reaction time is from about 30 minutes to about 4.5 hours. In an aspect, the alcohol stripping step is performed in vacuum. In an aspect, the alcohol stripping step is performed in vacuum, wherein nitrogen is injected through the bottom of the reactor and temperature is applied. In an aspect, the alcohol stripping step under vacuum is carried out at a temperature of from about 85° C. to about 95° C. for a time of from about 1 to 3 hours.

In a separate reaction of the parallel process, an acyl glyceride formation reaction b) is carried out by reacting a vegetal oil with glycerin to provide a mixture of monoacyl glycerides, diacyl glycerides, triacyl glycerides, residual glycerin and fatty acid. In an aspect, the acyl glyceride formation reaction is carried out in the presence of a basic catalyst. In an aspect, the basic catalyst is selected from a hydroxide-containing catalyst. In an aspect, the basic catalyst is selected from calcium hydroxide, sodium hydroxide, potassium hydroxide, and mixtures thereof. In an aspect, the basic catalyst is selected from an alkoxide-containing catalyst. In an aspect, the basic catalyst is sodium methoxide. In an aspect, the acyl glyceride formation reaction takes place at a temperature of from about 120° C. to about 180° C. In an aspect, the acyl glyceride formation reaction takes place in an inert gas. In an aspect, the acyl glyceride formation reaction takes place under a nitrogen sparge.

In an aspect, the vegetal oil used in the acyl glyceride formation reaction is selected from the group consisting of Soybean Oil, Canola Oil, Rapeseed Oil, Sunflower Oil, Corn Oil, and mixtures thereof. In an aspect, the vegetal oil is selected from the group consisting of Soybean Oil, Canola Oil, Rapeseed Oil, and mixtures thereof. In an aspect, the vegetal oil is Soybean Oil. In an aspect, the vegetal oil is Canola Oil. In an aspect, the vegetal oil is Rapeseed Oil. In an aspect, the vegetal oil has an Iodine Value of from about 80 to about 140 cg I/g.

The mixture of monoacyl glycerides (MAG), diacyl glycerides (DAG), and triacyl glycerides (TAG) composition of the acyl glyceride formation reaction b) is reacted in an acetylation reaction c) with acetic anhydride to form an acetylated glyceride composition comprising from about 7 to about 35% wt di-acylated monoglycerides and from about 45 to about 60% w mono-acylated diglycerides. In an aspect, the acetylation reaction is carried out in the presence of a catalyst. In an aspect, the catalyst is selected from methanosulfonic acid and sulfuric acid. In an aspect, the acetylation reaction is not catalyzed. It has been found that in some cases that compositions prepared using a separate acetylation catalyst can exhibit undesirable color properties.

In an aspect, the acetylation reaction takes place at a temperature of from about 60° C. to about 120° C. In an aspect, the acetylation reaction takes place at a temperature of about 85° C. In an aspect, the acetylation reaction time is from about 1 to 8 hours. In an aspect, the acetylation reaction time is from about 2 to about 4 hours.

In aspect, the acetylation reaction is followed by an acetic acid stripping step to remove any undesired acetic acid present in the reaction composition.

In an aspect, the acetic acid stripping step is carried out at a temperature of from about 100° C. to about 180° C. In an aspect, the acetic acid stripping step is carried out at a temperature of from about 135° C. to about 150° C. In an aspect, the acetic acid stripping step reaction time is from about 1 hour to about 7 hours. In an aspect, the acetic acid stripping step reaction time is from about 2 hours to about 6 hours.

In an aspect, the product of the acetylation reaction may be further treated by application of full vacuum steam to reduce the residual triacetin. In an aspect, the product of the acetylation reaction may be further treated by filtration, such as by use of celite and B80 clays, to reduce metals and polar impurities.

The C4-C10 fatty ester composition from the transesterification reaction a) is mixed with the acetylated glyceride composition from the acetylation reaction c) in a weight ratio of 3:7 to 7:3 to form an acetylated glyceride/fatty ester plasticizer composition.

In an aspect, the acetylated glyceride/fatty ester plasticizer composition has a C4-C10 fatty ester/acetylated glyceride weight ratio of 3:7 to 1:1. In an aspect, the acetylated glyceride/fatty ester plasticizer composition has a C4-C10 fatty ester/acetylated glyceride weight ratio of 3:7 to 6:7. In an aspect, the acetylated glyceride/fatty ester plasticizer composition has a C4-C10 fatty ester/acetylated glyceride weight ratio of 3:7 to 5:7. It has been found that epoxidized acetylated glyceride/fatty ester plasticizer compositions wherein the fatty ester component is present in an amount by weight that is the same or greater than the amount by weight of the acetylated glyceride is particularly advantageous for applications where the plasticizer desirably is used in environments where low VOC emissions is important.

In an aspect, the acetylated glyceride/fatty ester plasticizer composition has a C4-C10 fatty ester/acetylated glyceride weight ratio of 1:1 to 7:3. In an aspect, the acetylated glyceride/fatty ester plasticizer composition has a C4-C10 fatty ester/acetylated glyceride weight ratio of 7:6 to 7:3. In an aspect, the acetylated glyceride/fatty ester plasticizer composition has a C4-C10 fatty ester/acetylated glyceride weight ratio of 7:5 to 7:3. It has been found that epoxidized acetylated glyceride/fatty ester plasticizer compositions wherein the fatty ester component is present in an amount by weight that is the same or less than the amount by weight of the acetylated glyceride is particularly advantageous for applications where the plasticizer desirably is used in environments where a high plasticization effect is important.

In an aspect, the C4-C10 fatty ester composition and the acetylated glyceride composition are epoxidized before they are mixed together. In an aspect, the C4-C10 fatty ester composition and the acetylated glyceride composition are epoxidized after they are mixed together. In either approach, the final product is an epoxidized acetylated glyceride/fatty ester plasticizer composition.

Whether the C4-C10 fatty ester composition and the acetylated glyceride composition are to be epoxidized together or separately, the material to be epoxidized is reacted with hydrogen peroxide in the presence of catalyst. In an aspect, the catalyst is selected from phosphoric acid, formic acid or acetic acid. Once all the hydrogen peroxide is added and the double bonds are consumed the product is water washed multiple times to remove residual acid and peroxide before being dried under vacuum.

In an aspect, the epoxidation reaction is carried out at a temperature of from about 50° C. to about 100° C. In an aspect, the epoxidation reaction is carried out at a temperature of from about 55° C. to about 75° C. In an aspect, the epoxidation reaction time is from about 1 to about 5 hours. In an aspect, the epoxidation reaction time is from about 1.5 to about 3 hours.

Advantageously, in an aspect the epoxidized acetylated glyceride/fatty ester plasticizer composition comprises only a limited amount of fatty acid ethyl ester compounds, if such compounds are present at all. These compounds have been found to be undesirable components in a plasticized polymer product, because fatty acid ethyl ester compounds tend to be lost from the plasticized polymer through volatilization, extraction or migration. In an aspect, the epoxidized acetylated glyceride/fatty ester plasticizer composition comprises from 0 to about 3% wt of epoxidized fatty acid ethyl ester. In an aspect, the epoxidized acetylated glyceride/fatty ester plasticizer composition comprises from 0 to about 1% wt of epoxidized fatty acid ethyl ester. In an aspect, the epoxidized acetylated glyceride/fatty ester plasticizer composition comprises from 0 to about 0.5% wt of epoxidized fatty acid ethyl ester. In an aspect, the epoxidized acetylated glyceride/fatty ester plasticizer composition comprises from 0 to about 0.1% wt of epoxidized fatty acid ethyl ester.

Advantageously, in an aspect the epoxidized acetylated glyceride/fatty ester plasticizer composition comprises only a limited amount of fatty acid methyl ester compounds, if such compounds are present at all. These compounds have been found to be undesirable components in a plasticized polymer product, because fatty acid methyl ester compounds tend to be lost from the plasticized polymer through volatilization, extraction or migration. In an aspect, the epoxidized acetylated glyceride/fatty ester plasticizer composition comprises from 0 to about 3% wt of epoxidized fatty acid methyl ester. In an aspect, the epoxidized acetylated glyceride/fatty ester plasticizer composition comprises from 0 to about 1% wt of epoxidized fatty acid methyl ester. In an aspect, the epoxidized acetylated glyceride/fatty ester plasticizer composition comprises from 0 to about 0.5% wt of epoxidized fatty acid methyl ester. In an aspect, the epoxidized acetylated glyceride/fatty ester plasticizer composition comprises from 0 to about 0.1% wt of epoxidized fatty acid methyl ester.

Advantageously, in an aspect the epoxidized acetylated glyceride/fatty ester plasticizer composition comprises only a limited amount of triacetin. This compound has been found to be undesirable components in a plasticized polymer product, because triacetin tends to be lost from the plasticized polymer through volatilization, extraction or migration. In an aspect, the epoxidized acetylated glyceride/fatty ester plasticizer composition comprises from 0 to about 8% wt of triacetin. In an aspect, the epoxidized acetylated glyceride/fatty ester plasticizer composition comprises from 0 to about 5% wt of triacetin. In an aspect, the epoxidized acetylated glyceride/fatty ester plasticizer composition comprises from 0 to about 3% wt of triacetin. In an aspect, the epoxidized acetylated glyceride/fatty ester plasticizer composition comprises from 0 to about 1% wt of triacetin.

In an aspect, the epoxidized acetylated glyceride/fatty ester plasticizer composition is substantially free of dioctyl phthalate (DOP), di-isononyl phthalate (DINP), dioctil terephthalate (DOTP) and/or dioctyl cyclohexanoate (DOCH).

In an aspect, the epoxidized acetylated glyceride/fatty ester plasticizer composition has an epoxy oxygen content sufficiently high to provide compatibility of the plasticizer in the material to be plasticized, in particular in PVC. In an aspect, the epoxidized acetylated glyceride/fatty ester plasticizer composition has an epoxy oxygen content of from about 4 to about 8%. For purposes of the present discussion, epoxy oxygen content is determined by the test described in ASTM D1652-11.

Advantageously, in an aspect the epoxidized acetylated glyceride/fatty ester plasticizer composition has a residual hydroxyl content that is sufficiently low to prevent cross-reactions and so that the plasticizer is compatible with the material to be plasticized. In an aspect, the epoxidized acetylated glyceride/fatty ester plasticizer composition has a residual hydroxyl content of from 0 to about 40 mg KOH/g sample. In an aspect, the epoxidized acetylated glyceride/fatty ester plasticizer composition has a residual hydroxyl content of from 0 to about 30 mg KOH/g sample. In an aspect, the epoxidized acetylated glyceride/fatty ester plasticizer composition has a residual hydroxyl content of from 0 to about 20 mg KOH/g sample. For purposes of the present invention, residual hydroxyl content is determined by the test described in ASTM E1899-02.

Advantageously, in an aspect the epoxidized acetylated glyceride/fatty ester plasticizer composition has a metal content that is sufficiently low to prevent adverse reactions and/or adverse electrical conductivity of the final plasticized product. In an aspect, the epoxidized acetylated glyceride/fatty ester plasticizer composition has a metal content of from 0 to about 10 ppm. In an aspect, the epoxidized acetylated glyceride/fatty ester plasticizer composition has a metal content of from 0 to about 5 ppm. In an aspect, the epoxidized acetylated glyceride/fatty ester plasticizer composition has a metal content of from 0 to about 2 ppm. For purposes of the present invention, metal content is determined by inductively coupled plasma optical emission spectrometry (ICP-OES).

Advantageously, in an aspect the epoxidized acetylated glyceride/fatty ester plasticizer composition has a viscosity that facilitates mixing of the plasticizer with the material to be plasticized to provide effective distribution of the plasticizer throughout the material to be plasticized. It has been found that by selection of ingredients in the final plasticizer, the blended viscosity of the various ingredients can provide excellent overall mixability. In an aspect, the epoxidized acetylated glyceride/fatty ester plasticizer composition has a viscosity of from about 20 to about 100 cP at 25° C. In an aspect, the epoxidized acetylated glyceride/fatty ester plasticizer composition has a viscosity of from about 30 to about 700 cP at 25° C. For purposes of the present invention, viscosity is determined by analysis using a Brookfield Viscometer, #18 spindle at 200 RPM at 25° C.

Advantageously, in an aspect the epoxidized acetylated glyceride/fatty ester plasticizer composition has a favorably low APHA color value (sometimes referred to as the Hazen color value). For purposes of the present invention, the APHA color value is determined by the test described in ASTM D1209 and has the units “mg Pt/fl.” Alternatively, color may be measured using the Standard Test Method for Color of Transparent Liquids (Gardner Color Scale),” 2010, http://www.astm.org/Standards/D1544.htm.

In an aspect, the epoxidized acetylated glyceride/fatty ester plasticizer composition has an APHA color value of from 0 to about 150 mg Pt/fl. In an aspect, the epoxidized acetylated glyceride/fatty ester plasticizer composition has an APHA color value of from 0 to about 100 mg Pt/fl. In an aspect, the epoxidized acetylated glyceride/fatty ester plasticizer composition has an APHA color value of from 0 to about 70 mg Pt/fl.

Advantageously, in an aspect the epoxidized acetylated glyceride/fatty ester plasticizer composition has a glycerin content of from 0 to about 1% wt; or from 0 to about 0.5% wt; or from 0 to about 0.1% wt. For purposes of the present invention, the glycerin content of the composition is determined by the test described in ASTM D6584.

In an aspect, the epoxidized acetylated glyceride/fatty ester plasticizer composition may comprise an additional component selected from any additive useful for plastic compositions, for instance a stabilizer, antifogging agents, surfactants, biocides, fillers, slip agents, release agents, thickeners, lubricants, flow modifiers or processing aids, impact modifiers, pigments, viscosity reducers, flame retardants and diluent, or mixtures thereof.

In an aspect, the epoxidized acetylated glyceride/fatty ester plasticizer composition as described herein is mixed with an appropriate polymer resin, such as PVC, in an amount effective to provide a plasticizing effect. In an aspect, the epoxidized acetylated glyceride/fatty ester plasticizer composition is provided in a dry-blend format for use in suspension PVC applications. In an aspect, the epoxidized acetylated glyceride/fatty ester plasticizer composition is provided in an emulsion for use in plastisol PVC applications. In an aspect, the epoxidized acetylated glyceride/fatty ester plasticizer composition is mixed with an appropriate polymer resin at a mix ratio of from about 25 to about 300 PHR. In an aspect, the epoxidized acetylated glyceride/fatty ester plasticizer composition is mixed with an appropriate polymer resin at a mix ratio of from about 50 to about 150 PHR. In an aspect, the polymer resin comprises PVC.

The final plasticized polymer product (e.g. PVC products) can be applied for the manufacturing or processing of flexible and semi-rigid polymer articles (e.g. PVC articles) for many industries, such as for footwear, child care articles (i.e. toys), wires & cables, sealants & adhesives, coated fabric (i.e. synthetic leather and tarpaulins), flooring and medical devices (i.e. tubing and bags), among others.

### EXAMPLES

Epoxidized acetylated glyceride/fatty ester plasticizers were prepared as described herein.

### Example 1—Manufacturing Route 1

A plasticizer is prepared by a series of reactions with soybean oil, wherein the soybean oils are epoxidized prior to transesterification and acetylation.

The Route 1 comprises the following steps that will be detailed below:

(Step 1.1) Epoxidation of the vegetal oil (e.g., soybean oil);

(Step 1.2) Partial transesterification reaction;

(Step 1.3) Acetylation reaction; and

(Step 1.4) Optional Bleaching Step.

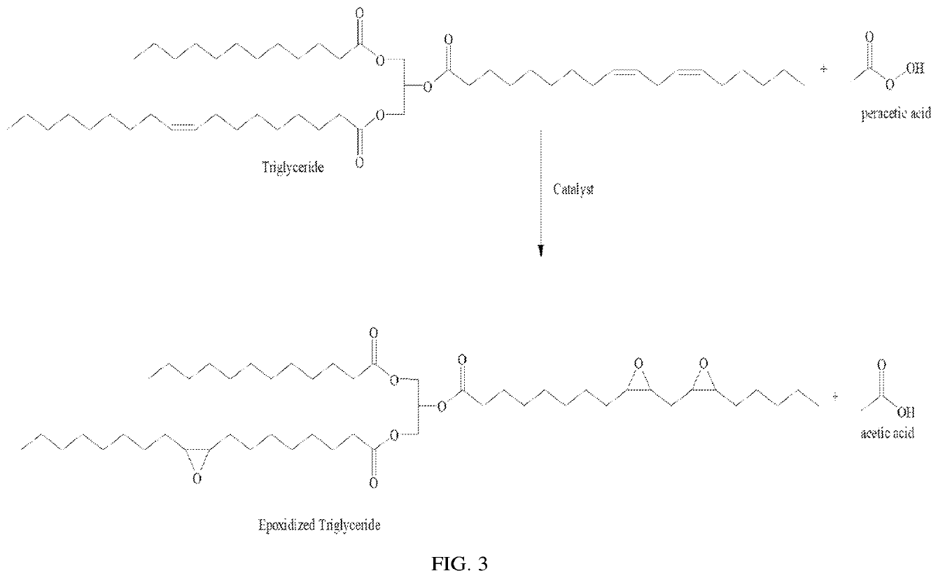

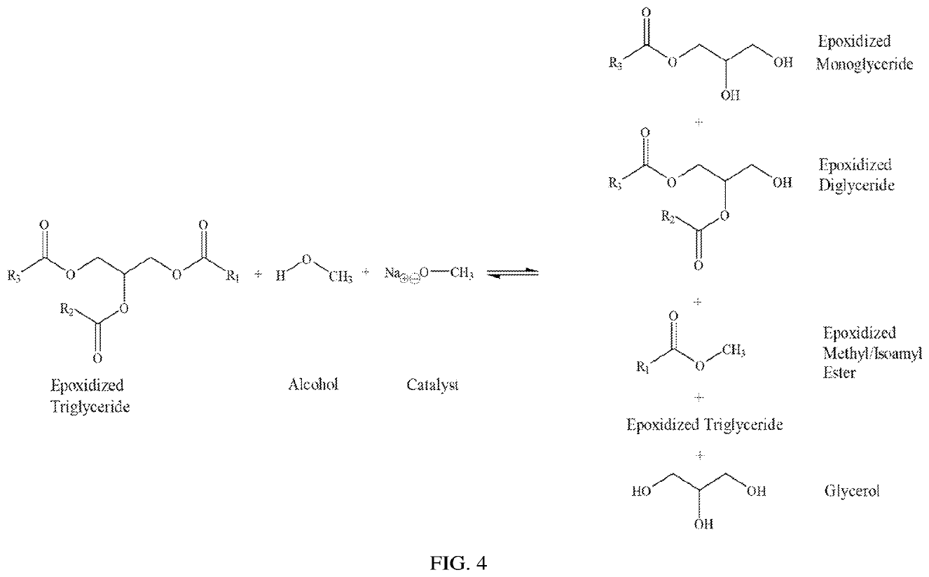

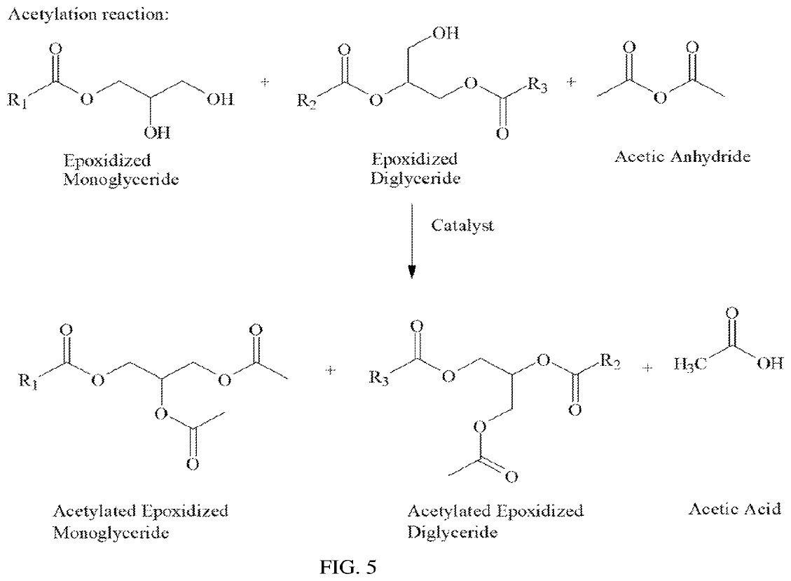

An illustration of chemical reactions of Steps 1.1-1.3 are shown in FIGS. 3, 4 and 5.

In an aspect, the partial transesterification reaction is carried out to have a final isopentyl fatty ester content of about 66% as a target. In an aspect, the product from this route is a partially transesterified isopentyl ester of epoxidized soybean oil. In an aspect, the product from this route is partially transesterified to provide a composition having a final isopentyl fatty ester content of from about 60% to about 90%. In an aspect, the product from this route is partially transesterified to provide a composition having a final isopentyl fatty ester content of from about 60% to about 70%. Plasticizers made by this process are referred to herein as “Plasticizer A.” Details of each step are described below:

### (Step 1.1) Epoxidation of the Vegetal Oil:

The raw material used is soybean oil.

The epoxidation is a reaction of the unsaturated portions of the vegetal oil with hydrogen peroxide in the presence of the phosphoric acid catalyst and forming the epoxy ring, with the generation of water as a by-product. The reaction is carried out at 65° C., with constant hydrogen peroxide flow for 2 hours. The reaction is controlled by monitoring the iodine index.

### (Step 1.2) Partial Transesterification Reaction:

An epoxidized triglyceride was reacted with isopentylic alcohol in the presence of a sodium methoxide NaOCH₃ catalyst at 65° C. for 2 hours and 20 minutes, followed by a second stage of transesterification at 65° C. for 1 hour and 11 minutes. This reaction results in epoxidized mono and di-glyceride and also in epoxidized isopentyl ester and glycerol.

During this step an alcohol stripping is also conducted. It is performed in vacuum, wherein nitrogen is injected through the bottom of the reactor and temperature is applied. Temperature is 130° C. and the reaction time is 4 hours and 15 minutes.

### (Step 1.3) Acetylation Reaction:

The epoxidized mono and di-glyceride and epoxidized isopentyl ester product of Step 1.2 is reacted with acetic anhydride. Specifically, the hydroxyl functionality present in the monoglycerides and diglycerides reacted with acetic anhydride, forming acetylated monoglycerides and diglycerides and acetic acid as a byproduct. The reaction is controlled by monitoring the hydroxyl value. Temperature is 85° C. and reaction time is 2 hours and 40 minutes.

During this step acetic acid stripping is carried out at temperature of 139° C. and the time of the acetic acid stripping reaction is 2 hours and 10 minutes.

### (Step 1.4) Bleaching Step:

The epoxidized acetylated glyceride/fatty ester plasticizer composition prepared in Step 1.3 is bleached by increasing the pH of the composition to above pH 9 by mixing with a 0.1% (in average) sodium hydroxide 50% aqueous solution.

The mass balance of illustrative batches and the detailed process steps are showed in Table 1 below.

TABLE 1
Mass Balance

| Mass (g) | |
|---|---|
| Transesterification reaction | |
| ESO | 12692.1 |
| Alcohol (C5) | 3326.6 |
| Sodium methoxide | 225.1 |
| Glycerin | 551.3 |
| Phosphoric Acid | 8.02 |
| Distilled (Alcohol)* | 1532.6 |
| Samples | 380.2 |
| Flush + loss | 1062.2 |
| Final product | 12897.8 |
| Final Yield - oil based | 101.62 |

Acetylation Reaction

| | |
|---|---|
| Partial ester | 5876.5 |
| Acetic Anhydride | 1471.2 |
| Methanosulfonic acid | 5.7 |
| Distilled (Acetic acid)* | 1095.6 |
| Sodium hydroxide | 5.71 |
| Hydrogen Peroxide | 58.9 |
| Samples | 212.3 |
| Flush + loss | 1583.9 |
| Final product | 5123.2 |
| Final Yield (%) ester based | 87.18 |

In a first batch, a composition denoted “Plasticizer A-1” is provided comprising isopentyl fatty ester, wherein the reaction conversion was 69%. The Ester Conversion formula is described below:

$$Ester Conversion ( \% ) = [ 1 - ( \frac{92 ( Glycerol MW ) * Sample total Glycerol ( \% )}{890 ( Oil MW )} ) ] * 100$$

The methodology used to measure sample's total glycerol is ASTM D6584.

In an aspect, ester content is measured by liquid chromatography. It has been found that gas chromatograph may be inaccurate because most of epoxidized oil is retained in the GC column, leading to false results.

Three (3) additional batches of compositions (Plasticizers A-2, A-3 and A-4) were prepared using the same process and formula conditions, providing compositions having an isopentyl fatty ester content of 59%. In an additional batch (Plasticizer A-5) the isopentyl fatty ester content was 85% ester content. Except for this batch the isopentyl fatty ester content range was between 59 to 69%.

### Example 2—Manufacturing Route 2

A plasticizer is prepared by a series of reactions with soybean oil, wherein the epoxidation step is carried out last in the sequence of reactions. It has been found that carrying out these process steps in this order provides many benefits, including increased yield, color reduction and reduced cost as compared to Route 1/Example 1. Moreover, because the color of the plasticizer prepared in this manner is lower as compared to Route 1/Example 1, it is possible to prepare an acceptable plasticizer product without a bleaching step. This provides particular benefit in reducing the number of reactants and improving yield of the final product.

An illustration of chemical reactions of Route 2 is shown in FIGS. 1-3.

Manufacturing Route 2 comprises the following steps that will be detailed below:

(Step 2.1) Partial Transesterification from vegetal oil;

(Step 2.2) Acetylation; and

(Step 2.3) Epoxidation.

The final products from this route (Route 2) are identified as “Plasticizer B.” The table below indicates the amount of each compound in an illustrative plasticizer composition obtained by this route, analyzed after the partial transesterification step:

TABLE 2
plasticizer composition
Plasticizer B

| | |
|---|---|
| Isopentyl ester | 78% |
| Monoglycerides | 6,8% |
| Diglycerides | 10,7% |
| Triglycerides | 3,1% |
| Glycerol | 0,8% |

(Step 2.1) Partial Transesterification from Vegetal Oil:

A triglyceride (3565 g of soybean oil) is reacted with 957.9 g pf isopentylic alcohol, in the presence of a 41.8 g of sodium methoxide Na(CH₃O) catalyst at 65° C. during 2 hours. This reaction results in mono and di-glyceride and also in isopentyl ester and glycerol.

During this step an alcohol stripping is also conducted. Alcohol stripping, for example, may be performed in vacuum, wherein nitrogen is injected through the bottom of the reactor and temperature is applied. Temperature: 85° C./Time of reaction: 2 hours.

### (Step 2.2) Acetylation Reaction:

466.2 g of acetic anhydride is reacted with the hydroxyls present in the monoglycerides and diglycerides in the presence of a 6.06 g of methanosulfonic acid catalyst, forming acetylated monoglycerides and diglycerides, and acetic acid as a byproduct. The end of the reaction is controlled by monitoring the hydroxyl value. Temperature 85° C./Time of reaction: 3 hours.

During this step an acetic acid stripping can be carried out at temperature 140° C. for a reaction time of 5 hours.

### (Step 2.3) Epoxidation:

The acetylated glyceride/isopentyl fatty ester intermediate prepared in Step 2.2 is epoxidized.

Specifically, the acetylated glyceride/isopentyl fatty ester intermediate is reacted with hydrogen peroxide in the presence of the phosphoric acid catalyst to form epoxy rings, with the generation of water as a by-product. The reaction is carried out at 65° C., with constant hydrogen peroxide flow for 2 hours. The reaction may be controlled by monitoring the iodine index.

### Example 3—Manufacturing Route 3

The Route 3 comprises the following steps:
(Step 3.1) transesterification reaction a) to prepare a C4-C10 fatty ester composition by reacting a C5 alcohol with a methyl ester of an unsaturated fatty acid (soybean oil residue).
(Step 3.2) acyl glyceride formation reaction b) by reacting a soybean oil with glycerin to provide a mixture of monoacyl glycerides, diacyl glycerides, triacyl glycerides, residual glycerin and fatty acid.
(Step 3.3) acetylation reaction c) of the mixture of monoacyl glycerides, diacyl glycerides, triacyl glycerides, residual glycerin and fatty acid;
(Step 3.4) Mixing the C4-C10 fatty ester composition of step 3.1 with the acetylated glyceride mixture of Step 3.3 in a ratio of (7:3); and
(Step 3.5) epoxidation of the mixture of Step 3.4.

### Example 4—Manufacturing Route 4

The Route 4 manufacturing process comprises the following steps that will be described in detail below:
(Step 4.1) transesterification reaction a). to prepare a C4-C10 fatty ester composition by reacting a C5 alcohol with a methyl ester of an unsaturated fatty acid (soybean oil residue);
(Step 4.2) epoxidation of the C4-C10 fatty ester composition of Step 4.1;
(Step 4.3) acyl glyceride formation reaction b) by reacting a soybean oil with glycerin to provide a mixture of monoacyl glycerides, diacyl glycerides, triacyl glycerides, residual glycerin and fatty acid;
(Step 4.4) acetylation reaction c) of the mixture of monoacyl glycerides, diacyl glycerides, triacyl glycerides, residual glycerin and fatty acid;
(Step 4.5) epoxidation of the mixture of Step 4.4; and
(Step 4.6) Mixing the epoxidized C4-C10 fatty ester composition of step 4.2 with the epoxidized acetylated glyceride mixture of Step 4.5.

### (Step 4.1) Transesterification Reaction a)

Three different soyate esters were prepared as follows:

### A) Preparation of Isopentyl Soyate

4756 g of soy methyl ester and 2885 g of isopentyl alcohol were charged to a 12 L 4-neck round bottom flask with mechanical stirring. The reactor was heated with a heating mantle to 70° C. while under a nitrogen sparge with a distillation condenser. The alcohol and ester were dried under vacuum to moisture <300 ppm. After the moisture content is below 300 ppm, 38 g of 25% methanolic sodium methoxide were added to the reactor and vacuum was applied in a step wise manner over 5 hours down to 40 Torr. Conversion of the ester was tracked by GC. Isopentyl ester content was greater than 98%, the reaction composition was re-pressurized and neutralized with 20.4 g of 98% phosphoric acid. After neutralization, the excess alcohol was distilled under 10 Torr vacuum at 160° C. until the hydroxyl value is less than 5 mg KOH/g. Finally, the product was cooled and filtered through celite and Pure Flo B 80 clay in order to remove the catalyst salt. The end product was 98.9% isopentyl soyate, 1.1% soy methyl ester.

### B) Iso-Nonyl Soyate

1620 g of soy methyl ester and 1602 g of iso-nonyl alcohol were charged to a 5 L 4-neck round bottom flask with mechanical stirring. The reactor was heated with a heating mantle to 70° C. while under a nitrogen sparge with a distillation condenser. The alcohol and ester were dried under vacuum to moisture <300 ppm. After the moisture <300 ppm, 17.1 g of 25% methanolic sodium methoxide were added to the reactor and vacuum was applied in a step wise manner over 5 hours down to 10 Torr. Conversion of the ester was tracked by GC. Once iso-nonyl ester content was greater than 98%, the reaction composition was re-pressurized and neutralized with 9.1 g of 98% phosphoric acid. After neutralization, the excess alcohol was distilled under 10 Torr vacuum at 205° C. until the hydroxyl value is <5 mg KOH/g. Finally, the product was cooled and filtered through celite and Pure Flo B80 clay in order to remove the catalyst salt. The end product was 99.2% iso-nonyl soyate, 0.8% soy methyl ester.

C) 2-ethyl hexyl soyate

1694 g of soy methyl ester and 1506 g of 2-ethyl hexyl alcohol were charged to a 5 L 4-neck round bottom flask with mechanical stirring. The reactor was heated with a heating mantle to 70° C. while under a nitrogen sparge with a distillation condenser. The alcohol and ester were dried under vacuum to moisture <300 ppm. After the moisture <300 ppm, 17 g of 25% methanolic sodium methoxide were added to the reactor and vacuum was applied in a step wise manner over 5 hours down to 15 Torr. Conversion of the ester was tracked by GC. Once 2-ethyl hexyl ester content was greater than 98%, the reaction composition was re-pressurized and neutralized with 9 g of 98% phosphoric acid. After neutralization, the excess alcohol was distilled under 10 Torr vacuum at 185° C. until the hydroxyl value is <5 mg KOH/g. Finally, the product was cooled and filtered through celite and Pure Flo B80 clay in order to remove the catalyst salt. The end product was 99.1% 2-ethyl hexyl soyate, 0.9% soy methyl ester.

### (Step 4.2) Epoxidation of the C4-C10 Fatty Ester Composition of Step 4.1.

The three different soyate esters prepared above were epoxidized as follows:

### A) Epoxy Isopentyl Soyate

3400 g of isopentyl soyate was charged to a 12 L round bottom flask along with 382 g of 88% formic acid. The reactor was heated with a heating mantle to 60° C. under nitrogen sparge. Once at temperature, 2750 g of 35% hydrogen peroxide was added drop-wise using an addition funnel over 6 hours and held at temperature until the Iodine Value was <5 cg I/g, while taking care to not let the product exotherm to >70° C. Once the Iodine value was 1.7 cg I/g, the reaction composition was cooled by adding cold water and allowed to separate. The organic layer was washed 4 times with 3 liters of cold water before being dried under vacuum at 60° C. and 5 Torr. The final product was 98.9% epoxy isopentyl soyate with an EOC of 5.53%, an IV 1.7 cg I/g, and a color of 235 APHA.

### B) Epoxidized Iso-Nonyl Soyate

1600 g of iso-nonyl soyate was charged to a 5 L round bottom flask along with 180 g of 88% formic acid. The reactor was heated with a heating mantle to 60° C. under nitrogen sparge. Once at temperature 1055 g of 35% hydrogen peroxide was added drop-wise using an addition funnel over 5 hours and held at temperature until the Iodine Value was <5 cg I/g, while taking care to not let the product exotherm to >70° C. Once the Iodine value was 3.2 cg I/g the reaction composition was cooled by adding cold water and allowed to separate. The organic layer was washed 4 times with 2 liters of cold water before being dried under vacuum at 60° C. and 5 Torr. The final product was 99.2% epoxy iso-nonyl soyate with an EOC of 4.95%, an IV 3.2 cg I/g, and a color of 209 APHA.

### C) Epoxidized 2-Ethyl Hexyl Soyate

1600 g of 2-ethyl hexyl soyate was charged to a 5 L round bottom flask along with 172 g of 88% formic acid. The reactor was heated with a heating mantle to 60° C. under nitrogen sparge. Once at temperature, 1200 g of 35% hydrogen peroxide was added drop-wise using an addition funnel over 6 hours and held at temperature until the Iodine Value was <5 cg I/g, while taking care to not let the product exotherm to >70° C. Once the Iodine value was 2.3 cg I/g, the reaction was cooled by adding cold water and was allowed to separate. The organic layer was washed 4 times with 2 liters of cold water before being dried under vacuum at 60° C. and 5 Torr. The final product was 99.1% epoxy 2-ethyl hexyl soyate with an EOC of 5.06%, an IV 2.3 cg I/g, and a color of 215 APHA.

(Step 4.3) Acyl Glyceride Formation Reaction b) by Reacting a Soybean Oil with Glycerin to Provide a Mixture of Monoacyl Glycerides, Diacyl Glycerides, Triacyl Glycerides, Residual Glycerin and Fatty Acid.

Three different acyl glyceride compositions were prepared having different relative amounts of monoacyl glyceride, diacyl glyceride and triacyl glyceride content as follows:

### A) High MAG Glyceride Composition

819 g of RBD soybean oil, 378 g of glycerin, and 1.14 g of calcium hydroxide were loading into a 2 liter reactor with mechanical stirring. The reactor was heated with a heating mantle to 230° C. while under nitrogen sparge with a distillation condenser. Once at temperature, glyceride conversion was monitored by GC. When >60% monoglyceride content was achieved, 1.39 g of phosphoric acid was added and the reactor was cooled to 205° C. Once at 205° C., 20 Torr of vacuum was applied to distill excess glycerin to a glycerin content of less than 3%.

### B) High DAG Glyceride Composition

851 g of RBD soybean oil, 73.7 g of technical grade glycerin, and 2.2 g of calcium hydroxide were added to a 4 neck 2 L flask with mechanical stirring. The reactor was heated with a heating mantle to 150° C. while under nitrogen sparge with a distillation condenser. Once at temperature, glyceride conversion was monitored by GC until distribution equilibrium was reached. The resulting composition had a diacyl glyceride content of 54.3% diacyl glyceride.

### C) High TAG Glyceride Composition

101.5 kg of RBD soybean oil, 3.7 kg of technical grade glycerin, and 250 g of solid sodium methoxide were added to a 210 L reactor with mechanical stirring. The reactor was heated with a heating mantle to 150° C. while under nitrogen sparge with a distillation condenser. Once at temperature, glyceride conversion was monitored by GC until distribution equilibrium was reached. The resulting composition had a triacyl glyceride content of 35.7% Triglyceride.

### (Step 4.4) Acetylation Reaction c) of the Mixture of Monoacyl Glycerides, Diacyl Glycerides, Triacyl Glycerides, Residual Glycerin and Fatty Acid;

The three acyl glyceride compositions prepared in Step 4.3 above were acetylated as follows:

### A) High MAG Acetylated Glyceride Composition

Once residual glycerin in the High MAG Glyceride composition prepared above was <3%, the reactor was re-pressurized with nitrogen and cooled to 110° C. At 110° C., 580 g of acetic anhydride were added dropwise over 1 hour while care was taken not to let the exotherm exceed 120° C. Once the addition was complete the reaction was held at 120° C. until the hydroxyl peak in the IR disappeared. When the OH peak was gone the reactor was heated to 160° C. under nitrogen sparge; then 10 torr vacuum was applied in a step wise manner to remove excess acetic acid and anhydride. When the Acid Value was less than 1 mgKOH/g, the reactor was re-pressurized with nitrogen and cooled. Acid Value is measured in accordance with AOCS Method Cd 3d-63. Finally, the product was filtered using Pure Flo B80 clay and celite in order to remove catalyst salts. The final product was 61.8% acetylated monoglyceride, 36.9% acetylated diglyceride and 1.1% triglyceride.

### B) High DAG Acetylated Glyceride Composition

The High DAG Glyceride Composition prepared above was cooled to 110° C. 274 g of acetic anhydride was then added dropwise over 1 hour while care was taken not to let the exotherm exceed 120° C. Once the addition was complete, the reaction was held at 120° C. until the hydroxyl peak in the FTIR disappeared. When the OH peak was gone the reactor was heated to 160° C. under nitrogen sparge, then 10 torr vacuum was applied in a step-wise manner to remove excess acetic acid and anhydride. When the Acid Value was less than 1 mgKOH/g, the reactor was re-pressurized with nitrogen and cooled. Finally, the product was filtered using Pure Flo B80 clay and celite in order to remove catalyst salts. The final product was 29% acetylated monoglyceride, 54.3% acetylated diglyceride, 15.8% triglyceride, and 0.9% triacetin.

### C) High TAG Acetylated Glyceride Composition

The High TAG Glyceride Composition prepared above was cooled to 110° C. 13.8 kg of acetic anhydride was then added dropwise over 1 hour while care was taken not to let the exotherm exceed 120° C. Once the addition was complete, the reaction was held at 120° C. until the hydroxyl peak in the FTIR disappeared. When the OH peak was gone the reactor was heated to 160° C. under nitrogen sparge, then 10 torr vacuum was applied in a step-wise manner to remove excess acetic acid and anhydride. When the Acid Value was less than 1 mgKOH/g, the reactor was re-pressurized with nitrogen and cooled. Finally, the product was filtered using Pure Flo B80 clay and celite in order to remove catalyst salts. The final product was 11.2% acetylated monoglyceride, 53% acetylated diglyceride, 35.7% triglyceride, and 0.1% triacetin.

### (Step 4.5) Epoxidation of the Mixture of Step 4.4.

### A) High MAG Acetylated Expoxidized Glyceride Composition

952 g of the High MAG Acetylated Glyceride Composition prepared above was charged to a 2 L round bottom flask along with 96 g of 88% formic acid. The reactor was heated with a heating mantle to 60° C. under nitrogen sparge. Once at temperature, 760 g of 35% hydrogen peroxide was added drop-wise using an addition funnel over 5 hours and held at temperature until the Iodine Value was <5 cg I/g, while taking care to not let the product exotherm to >70° C. Once the Iodine value was 2 cg I/g, the reaction composition was cooled by adding cold water and allowed to separate. The organic layer was washed 4 times with 2 liters of cold water before being dried under vacuum at 60° C. and 5 Torr. The final product had an Epoxide Oxygen Content of 4.93%, an IV 2 cg I/g, and a color of 44 APHA. Epoxide Oxygen Content is measured in accordance with ASTM D1652-04.

B) High DAG Acetylated Expoxidized Glyceride Composition

351 g of the High DAG Acetylated Glyceride Composition as prepared above was charged to a 1 L round bottom flask along with 37.2 g of 88% formic acid. The reactor was heated with a heating mantle to 60° C. under nitrogen sparge. Once at temperature, 283 g of 35% hydrogen peroxide was added drop-wise using an addition funnel over 4 hours and held at temperature until the Iodine Value was <5 cg I/g, while taking care to not let the product exotherm to >70° C. Once the Iodine value was 2 cg I/g, the reaction composition was cooled by adding cold water and allowed to separate. The organic layer was washed 4 times with 2 liters of cold water before being dried under vacuum at 60° C. and 5 Torr. The final product had an Epoxide Oxygen Content of 5.71%, an IV 1.8 cg I/g, and a color of 277 APHA.

C) High TAG Acetylated Expoxidized Glyceride Composition

700 g of the High TAG Acetylated Glyceride Composition was charged to a 2 L round bottom flask along with 90 g of 88% formic acid. The reactor was heated with a heating mantle to 60° C. under nitrogen sparge. Once at temperature, 605 g of 35% hydrogen peroxide was added drop-wise using an addition funnel over 6 hours and held at temperature until the Iodine Value was <5 cg I/g, while taking care to not let the product exotherm to >70° C. Once the Iodine value was 2 cg I/g, the reaction composition was cooled by adding cold water and allowed to separate. The organic layer was washed 4 times with 2 liters of cold water before being dried under vacuum at 60° C. and 5 Torr. The final product had an Epoxide Oxygen Content of 5.92%, an IV 1.9 cg I/g, and a color of 310 APHA.

(Step 4.6) Mixing the Epoxidized C4-C10 Fatty Ester Composition of Step 4.2 with the Epoxidized Acetylated Glyceride Mixture of Step 4.5.

Seven different blends of the above epoxidized C4-C10 fatty ester compositions of step 4.2 with the epoxidized acetylated glyceride mixture of Step 4.5 were prepared as follows:

Blend 1—595 g of 4.2A (Epoxy Isopentyl Soyate) was blended with 255 g of 4.5B (high DAG acetylated epoxy glyceride) at room temperature using mechanical agitation for 1 hour. The final product had an Acid Value 1.8 mg KOH/g, Epoxy Oxygen Content of 5.35%, Hydroxyl Value of 32 mg KOH/g, Iodine Value of 2.8 cg I/g, and a viscosity of 70 cP at 25° C.

Blend 2—425 g of 4.2A (Epoxy Isopentyl Soyate) was blended with 425 g of 4.5B (high DAG acetylated epoxy glyceride) at room temperature using mechanical agitation for 1 hour. The final product had an Acid Value 1.8 mg KOH/g, Epoxy Oxygen Content of 5.3%, Hydroxyl Value of 27 mg KOH/g, Iodine Value of 2.6 cg I/g, and a viscosity of 118 cP at 25° C.

Blend 3—595 g of 4.2A (Epoxy Isopentyl Soyate) was blended with 255 g of 4.5B (high DAG epoxy glyceride) at room temperature using mechanical agitation for 1 hour. The final product had an Acid Value 1.8 mg KOH/g, Epoxy Oxygen Content of 5.25%, Hydroxyl Value of 32 mg KOH/g, Iodine Value of 2.4 cg I/g, and a viscosity of 266 cP at 25° C.

Blend 4—425 g of 4.2A (Epoxy Isopentyl Soyate) was blended with 425 g of 4.5C (high TAG acetylated epoxy glyceride) at room temperature using mechanical agitation for 1 hour. The final product had an Acid Value of 1.5 mg KOH/g, Epoxy Oxygen Content of 5.92%, Hydroxyl Value of 19 mg KOH/g, Iodine Value of 2.5 cg I/g, and a viscosity of 123 cP at 25° C.

Blend 5—425 g of 4.2A (Epoxy Isopentyl Soyate) was blended with 425 g of 4.5A (high MAG acetylated epoxy glyceride) at room temperature using mechanical agitation for 1 hour. The final product had an Acid Value of 1.1 mg KOH/g, Epoxy Oxygen Content of 5.16%, Hydroxyl Value of 19 mg KOH/g, Iodine Value of 1.6 cg I/g, and a viscosity of 56 cP at 25° C.

Blend 6—560 g of 4.2B (Epoxy Isopentyl Soyate) was blended with 560 g of 4.5B (high DAG acetylated epoxy glyceride) at room temperature using mechanical agitation for 1 hour. The final product had an Acid Value of 1.1 mg KOH/g, Epoxy Oxygen Content of 5.16%, Hydroxyl Value of 23 mg KOH/g, Iodine Value of 2.6 cg I/g, and a viscosity of 94 cP at 25° C.

Blend 7—560 g of 4.2C (Epoxy 2-ethyl hexyl Soyate) was blended with 560 g of 4.5B (high DAG acetylated epoxy glyceride) at room temperature using mechanical agitation for 1 hour. The final product had an Acid Value of 1.3 mg KOH/g, Epoxy Oxygen Content of 5.11%, Hydroxyl Value of 23 mg KOH/g, an Iodine Value of 2.2 cg I/g, and a viscosity of 84 cP at 25° C.

The above blends were evaluated, and it was found that Blend 1 had the highest plasticizing efficiency. Blend 6 was found to have the lowest amount of VOC present in the formulation after 7 days at 100° C.

As used herein, the terms “about” or “approximately” mean within an acceptable range for the particular parameter specified as determined by one of ordinary skill in the art, which will depend in part on how the value is measured or determined, e.g., the limitations of the sample preparation and measurement system. Examples of such limitations include preparing the sample in a wet versus a dry environment, different instruments, variations in sample height, and differing requirements in signal-to-noise ratios. For example, “about” can mean greater or lesser than the value or range of values stated by 1/10 of the stated values, but is not intended to limit any value or range of values to only this broader definition. For instance, a concentration value of about 30% means a concentration between 27% and 33%. Each value or range of values preceded by the term “about” is also intended to encompass the embodiment of the stated absolute value or range of values. Alternatively, particularly with respect to biological systems or processes, the term can mean within an order of magnitude, preferably within 5-fold, and more preferably within 2-fold, of a value.

Throughout this specification and claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein “consisting of” excludes any element, step, or ingredient not specified in the claim element. When used herein, “consisting essentially of” does not exclude materials or steps that do not materially affect the basic and novel characteristics of the claim. In the present disclosure of various embodiments, any of the terms “comprising”, “consisting essentially of” and “consisting of” used in the description of an embodiment may be replaced with either of the other two terms.

All patents, patent applications (including provisional applications), and publications cited herein are incorporated by reference as if individually incorporated for all purposes. Unless otherwise indicated, all parts and percentages are by weight and all molecular weights are weight average molecular weights. The foregoing detailed description has been given for clarity of understanding only. No unnecessary limitations are to be understood therefrom. The invention is not limited to the exact details shown and described, for variations obvious to one skilled in the art will be included within the invention defined by the claims.

1. A process to produce a plasticizer composition, comprising:
a) reacting a vegetal oil with isopentyl alcohol to provide a glyceride/fatty ester composition comprising from about from about 40 to about 80% wt isopentyl fatty acid ester;
b) reacting the glyceride/fatty ester composition of step a) with acetic anhydride to form an acetylated glyceride/fatty ester composition; and
c) epoxidizing the acetylated glyceride/fatty ester composition of step b) to form an epoxidized acetylated glyceride/fatty ester plasticizer composition;
wherein the epoxidized acetylated glyceride/fatty ester plasticizer composition is free of phthalate.

2. (canceled)

3. The process of claim 1, wherein the epoxidized acetylated glyceride/fatty ester plasticizer composition comprises:
i) from about 40-80% wt of epoxidized fatty acid isopentyl ester and/or its isomers
ii) from about 5 to about 25% wt of acetylated and epoxidized monoglyceride or its isomers;
ii) from about 10 to about 30% wt of acetylated and epoxidized diglyceride and/or its isomers;
iii) from about 3 to about 20% wt of epoxidized triglyceride;
iv) from 0 to about 10% wt of epoxidized fatty acid ethyl ester;
v) from 0 to about 10% wt of epoxidized fatty acid methyl ester; and
vi) from 0 to about 8% wt of triacetin.

4. The process of claim 1, wherein the epoxidized acetylated glyceride/fatty ester plasticizer composition comprises:
i) from about 45-60% wt of epoxidized fatty acid isopentyl ester and/or its isomers
ii) from about 10 to about 20% wt of acetylated and epoxidized monoglyceride or its isomers;
ii) from about 15 to about 25% wt of acetylated and epoxidized diglyceride and/or its isomers;
iii) from about 5 to about 15% wt of epoxidized triglyceride;
iv) from 0 to about 5% wt of epoxidized fatty acid ethyl ester;
v) from 0 to about 5% wt of epoxidized fatty acid methyl ester; and
vi) from 0 to about 5% wt of triacetin.

5. The process of claim 1, wherein the vegetal oil is selected from the group consisting of Soybean Oil, Canola Oil, Rapeseed Oil, Sunflower Oil, Corn Oil, and mixtures thereof; or wherein the vegetal oil is selected from the group consisting of Soybean Oil, Canola Oil, Rapeseed Oil, and mixtures thereof; or wherein the vegetal oil is Soybean Oil; or wherein the vegetal oil is Canola Oil; or wherein the vegetal oil is Rapeseed Oil.

6. The process of claim 1, wherein the epoxidized acetylated glyceride/fatty ester plasticizer composition comprises from 0 to about 3% wt of epoxidized fatty acid ethyl ester; or wherein the epoxidized acetylated glyceride/fatty ester plasticizer composition comprises from 0 to about 1% wt of epoxidized fatty acid ethyl ester; or wherein the epoxidized acetylated glyceride/fatty ester plasticizer composition comprises from 0 to about 0.5% wt of epoxidized fatty acid ethyl ester; or wherein the epoxidized acetylated glyceride/fatty ester plasticizer composition comprises from 0 to about 0.1% wt of epoxidized fatty acid ethyl ester.

7. The process of claim 1, wherein the epoxidized acetylated glyceride/fatty ester plasticizer composition comprises from 0 to about 3% wt of epoxidized fatty acid methyl ester; or wherein the epoxidized acetylated glyceride/fatty ester plasticizer composition comprises from 0 to about 1% wt of epoxidized fatty acid methyl ester; or wherein the epoxidized acetylated glyceride/fatty ester plasticizer composition comprises from 0 to about 0.5% wt of epoxidized fatty acid methyl ester; or wherein the epoxidized acetylated glyceride/fatty ester plasticizer composition comprises from 0 to about 0.1% wt of epoxidized fatty acid methyl ester.

8. The process of claim 7, wherein the epoxidized acetylated glyceride/fatty ester plasticizer composition comprises from 0 to about 3% wt of triacetin; or wherein the epoxidized acetylated glyceride/fatty ester plasticizer composition comprises from 0 to about 1% wt of triacetin.

9. The process of claim 8, wherein the epoxidized acetylated glyceride/fatty ester plasticizer composition has an epoxy oxygen content of from about 4 to about 8%.

10. The process of claim 9, wherein the vegetal oil has an Iodine Value of from about 80 to about 140 cg I/g.

11. The process of claim 10, wherein the epoxidized acetylated glyceride/fatty ester plasticizer composition has a residual hydroxyl content of from 0 to about 40 mg KOH/g sample; or wherein the epoxidized acetylated glyceride/fatty ester plasticizer composition has a residual hydroxyl content of from 0 to about 30 mg KOH/g sample; or wherein the epoxidized acetylated glyceride/fatty ester plasticizer composition has a residual hydroxyl content of from 0 to about 20 mg KOH/g sample.

12. The process of claim 11, wherein the epoxidized acetylated glyceride/fatty ester plasticizer composition has a metal content of from 0 to about 10 ppm; or wherein the epoxidized acetylated glyceride/fatty ester plasticizer composition has a metal content of from 0 to about 5 ppm; or wherein the epoxidized acetylated glyceride/fatty ester plasticizer composition has a metal content of from 0 to about 2 ppm.

13. The process of claim 12, wherein the epoxidized acetylated glyceride/fatty ester plasticizer composition has a viscosity of from about 20 to about 100 cP at 25° C.; or wherein the epoxidized acetylated glyceride/fatty ester plasticizer composition has a viscosity of from about 30 to about 700 cP at 25° C.

14. The process of claim 1, wherein the epoxidized acetylated glyceride/fatty ester plasticizer composition has an APHA color value of from 0 to about 150 mg Pt/fl; or wherein the epoxidized acetylated glyceride/fatty ester plasticizer composition has an APHA color value of from 0 to about 100 mg Pt/fl; or wherein the epoxidized acetylated glyceride/fatty ester plasticizer composition has an APHA color value of from 0 to about 70 mg Pt/fl.

15. The process of claim 1, wherein the epoxidized acetylated glyceride/fatty ester plasticizer composition has a glycerin content of from 0 to about 1% wt; or the epoxidized acetylated glyceride/fatty ester plasticizer composition has a glycerin content of from 0 to about 0.5% wt; or the epoxidized acetylated glyceride/fatty ester plasticizer composition has a glycerin content of from 0 to about 0.1% wt.

16. (canceled)

17. An epoxidized acetylated glyceride/fatty ester plasticizer composition comprising
i) from about 40-80% wt of epoxidized fatty acid isopentyl ester and/or its isomers
ii) from about 5 to about 25% wt of acetylated and epoxidized monoglyceride or its isomers;
ii) from about 10 to about 30% wt of acetylated and epoxidized diglyceride and/or its isomers;
iii) from about 3 to about 20% wt of epoxidized triglyceride;
iv) from 0 to about 10% wt of epoxidized fatty acid ethyl ester;
v) from 0 to about 10% wt of epoxidized fatty acid methyl ester; and
vi) from 0 to about 8% wt of triacetin.

18. The epoxidized acetylated glyceride/fatty ester plasticizer composition of claim 16 or 17, wherein the epoxidized acetylated glyceride/fatty ester plasticizer composition comprises:
i) from about 45-60% wt of epoxidized fatty acid isopentyl ester and/or its isomers
ii) from about 10 to about 20% wt of acetylated and epoxidized monoglyceride or its isomers;
ii) from about 15 to about 25% wt of acetylated and epoxidized diglyceride and/or its isomers;
iii) from about 5 to about 15% wt of epoxidized triglyceride;
iv) from 0 to about 5% wt of epoxidized fatty acid ethyl ester;
v) from 0 to about 5% wt of epoxidized fatty acid methyl ester; and
vi) from 0 to about 5% wt of triacetin.

19. The epoxidized acetylated glyceride/fatty ester plasticizer composition of claim 18, wherein the epoxidized acetylated glyceride/fatty ester plasticizer composition has a viscosity of from about 20 to about 100 cP at 25° C.; or wherein the epoxidized acetylated glyceride/fatty ester plasticizer composition has a viscosity of from about 30 to about 700 cP at 25° C.

20. The epoxidized acetylated glyceride/fatty ester plasticizer composition of claim 19, wherein the epoxidized acetylated glyceride/fatty ester plasticizer composition has an APHA color value of from 0 to about 150 mg Pt/fl; or wherein the epoxidized acetylated glyceride/fatty ester plasticizer composition has an APHA color value of from 0 to about 100 mg Pt/fl; or wherein the epoxidized acetylated glyceride/fatty ester plasticizer composition has an APHA color value of from 0 to about 70 mg Pt/fl.

21. A plasticized polyvinyl chloride composition comprising the epoxidized acetylated glyceride/fatty ester plasticizer composition of claim 17.

22-43. (canceled)
